(19) United States
(12) Patent Application Publication
(10) Pub. No.: US 2010/0106070 A1
(43) Pub. Date: Apr. 29, 2010

(54) Joint bandage

(76) Inventors: Jens Schlomski, Duderstadt (DE); Peter Demuth, Dingelstadt (DE); Holger Reinhardt, Kempen (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/603,612

(22) Filed: Oct. 22, 2009

(30) Foreign Application Priority Data
Oct. 23, 2008 (DE) 10 2008 052 860.9

(51) Int. Cl. A61F 13/06

(52) U.S. Cl. 602/63

(57) ABSTRACT

The invention relates to a joint bandage having a tubular base body made of an elastic textile material with an insert in the area of the bend in the joint, such that the elasticity of the insert is greater than that of the base body, and two areas of differing elasticity are formed in the insert, their individual elasticities being greater than that of the base body.

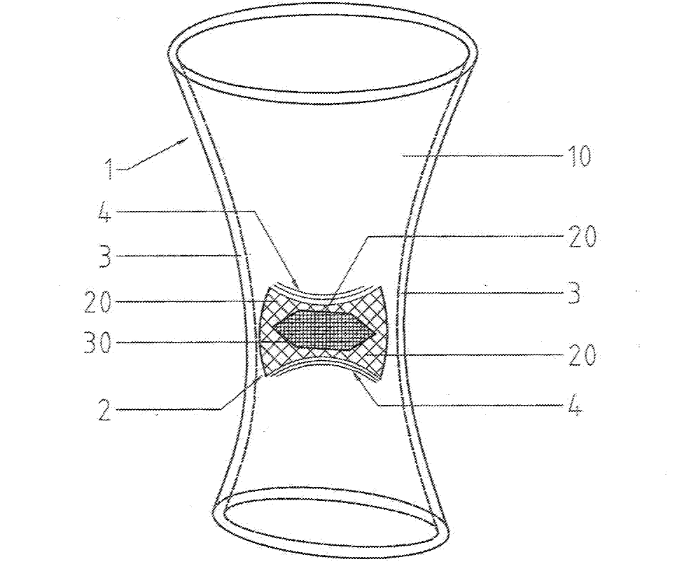

### BACKGROUND OF THE INVENTION

1. Field of the Invention

The invention relates to a joint bandage having a tubular base body made of an elastic textile material with an insert in the area of the bend, such that the elasticity of the insert is greater than that of the base body.

2. Background Description

The joint bandage is preferably to be used as a knee-joint bandage, but it may also be used as an elbow joint bandage, for example.

Joint bandages, especially knee-joint bandages, have long been known. DE 663 643 relates to a knee-joint bandage, which is to be used for so-called swinging joints or loose joints. The bandage serves to stabilize the knee joint. With knee-joint bandages, the problem arises that the front surface increases in flexion of the knee, while the popliteal fossa, i.e., the hollow of the knee, is reduced. The material of which a knee-joint bandage is made must conform to this movement. To prevent the material of the bandage from wrinkling at the hollow of the knee, the bandage is made of two different knit fabric parts, in which the knit fabric has been stretched to the greatest possible length in one direction and therefore is inelastic in this direction, whereas the knit fabric of the posterior part of the knee-joint bandage runs obliquely to the edges of the knee-joint bandage and is elastic in one direction and inelastic in the other direction.

DE 42 37 398 A1 describes a bandage for a knee-joint made of an elastic bandage material in tubular form having an anterior bandage part and a posterior bandage part and, if necessary, spring rods that run longitudinally and have been incorporated into the posterior bandage part. The bandage consists of an anatomically shaped tubular body with a circumferential insert of a highly elastic ribbed knit. A pad of a soft material or a soft-elastic material has been inserted into the posterior bandage part, acting on the muscle insertion points.

DE 43 22 028 C3 describes a joint bandage having a stocking-shaped tube of elastic, extensible textile material. The tube has an insert which is incorporated seamlessly into the joint area and has a greater extensibility in the longitudinal direction of the tube than in the circumferential direction of the tube and extends over no more than half of the circumference of the tube. The insert comprises a thinner, more elastic binding structure, which is different from that of the material having the tube, such that the insert is designed as an approximately lenticular intarsia knit area having an extensibility approximately twice as great as that of the tubular textile material.

DE 297 24 692 U1 describes a joint bandage having a knit as an insert part in a joint area having threads of differing elasticity. The knit fabric in the area of the hollow of the joint, in particular in the hollow of the knee is inserted seamlessly into the bandage and has bridging stitches which bridge two stitches and are provided in alternation on the two sides of the knit in the direction of knitting.

DE 10 2004 040 793 A1 describes an elastic knee-joint bandage made of an elastic textile material. To avoid wrinkling in the knee-joint, which is unpleasant for the wearer of the bandage, a highly elastic insert is incorporated into the textile material of the bandage in the area of the hollow of the knee in comparison with a profile insert. This insert stretches on extension of the knee and contracts again on flexion of the knee without thereby resulting in any significant wrinkling.

The object of the invention is to provide an improved joint bandage, in particular an improved knee-joint bandage, with which adequate stability of the joint is achieved on the one hand, while on the other hand a high wearing comfort can be provided.

According to the invention, this object is achieved by a joint bandage having the features of claim 1. Advantageous embodiments and further refinements of the invention are defined in the subclaims.

With the inventive joint bandage having a tubular base body made of an elastic textile material with an insert in the area of the bend, wherein the elasticity of the insert is greater than that of the base body, two areas of differing elasticity are formed in the insert, their individual elasticities each being greater than that of the base body. Through the design of the insert on the flexion side having multiple different elasticities, each being greater than that of the base body, it is possible to achieve an improved fit of the bandage material in the area of the flexed joint without thereby resulting in excessive wrinkling. Due to the differing elasticities within the insert, it is possible to provide an improved adaptation of the properties of the fabric in the area of the flexed joint, where there properties approximate the properties of skin, if possible.

According to a further embodiment of the invention, a central area is formed in the insert, with transition areas having a greater elasticity than the central area being connected at least medially and laterally to this central area. The central area is then surrounded at least laterally by transitional areas having a relatively greater elasticity than the central area, so there is a relatively fixed central area, which stabilizes the insert and prevents the hollow elastic fabric from bulging out in the transition areas. The moderately elastic central area thus stabilizes the insert and defines a preferential direction of the textile movement in flexion of the joint. This moderately elastic central area induces a compressed articular state, whereas the highly elastic transitional areas which can also completely surround the central area, prevent wrinkles from developing due to their high flexibility and extensibility. The central area may be designed so that a defined pressure may be exerted on the hollow of the knee, e.g., to provide a therapeutic massaging effect.

The central area may be designed to be essentially hexagonal or lenticular, such that the longest axis or the greatest width of the central area lies in the medial/lateral plane. The hexagon is thus a flattened and widened hexagon; with a rounded lenticular shape, the long axis lies essentially in the medial/lateral plane of the joint.

According to a further embodiment of the invention, the elasticity of the central area is greater in the medial/lateral direction than in the proximal/distal direction, to facilitate preflexion and to define a preferential direction of movement of the fabric in flexion of a joint.

The transitional areas may have a mesh structure, yielding a high degree of ventilation in the area of the bend in the joint, which ensures greater wearing comfort. At the same time, because of the mesh structure of the highly elastic fabric, whether knit or woven, a sufficient flexibility is provided in areas of the bandage that are flexed to a high degree, e.g., in the area of the tendon junctions, so it is possible to prevent an accumulation of material and wrinkling in these especially sensitive areas of the bend in the joint.

According to a further embodiment of the invention, the edges of the insert are designed with a curvature, so that the proximal and distal edges run with a curve toward the inside, and the medial and lateral edges run with a curve to the outside. Thus, there is a biconcave design in the proximal-distal direction, whereas a biconvex design of the edges is provided on the lateral edges. Such an embodiment of the insert is obtained in particular in the applied state of the bandage, but may also be achieved in the flat extended state. The design of the proximal and distal edges with flexion inwardly ensures an elasticity and design of the highly elastic areas in comparison with the base body, which is modeled on the natural flexion of the joint.

The insert may be incorporated into the base body, in particular being knit into it. This avoids interfering seams in the transition area from the base body to the insert in the flexion of the joint.

The joint bandage is preferably preflexed to increase wearing comfort. The tubular base structure may taper conically from each of its ends to obtain a shape approximating that of the respective extremity, which increases wearing comfort. Likewise, reinforcing struts may also be provided, preferably in the medial and lateral areas, to achieve an increased joint stability for the use of the bandage. Pads, e.g., ring pads to hold the patella or pressure pads for the elbow, may be provided on the extensor side and/or mediolaterally for an elbow bandage.

An exemplary embodiment of the present invention is explained in greater detail below with reference to the accompanying FIGURE. The FIGURE shows a schematic perspective view of a joint bandage in a posterior view.

The single FIGURE shows a joint bandage 1 in a posterior view. The joint bandage 1 has a base body 10, comprising a tubular elastic textile. The base body 10 may be stretched in the longitudinal direction as well as in the circumferential direction, the elasticity of the base body 10 being selected such that, on the one hand, sufficient stabilization of the joint in the applied state of the joint bandage is achieved, while on the other hand, the bandage 1 is securely held on the extremities, and furthermore, adequate wearing comfort is ensured without too greatly compressing the tissue surrounded by the bandage 1.

Reinforcing rods or spring rods 3 are incorporated into the sides of the joint bandage 1, i.e., in the mediolateral area, stabilizing the joint bandage 1 in its longitudinal extent and also supporting the bandaged joint. The reinforcing rods 3 are sufficiently flexible, so that flexion of the joint may occur with the least possible hindrance.

The base body 10 may have a conical widened area, at least toward one opening, for the case when it is used as a knee-joint bandage. For the case when it is used as a knee-joint bandage, a conically widened opening of the part of the bandage 1 coming in contact with the thigh is advisable in order to increase wearing comfort. The calf portion, i.e., the lower opening of the tubular base body 10, may be cylindrical or may also be designed with an area that widens conically in the direction of the opening. It is likewise possible for the joint bandage 1 to be preflexed, i.e., to be preshaped in the direction of flexion.

An elastic insert 2 having a central area 30 is provided in the flexion area of the joint bandage 1, namely in the area of the hollow of the knee in the case of a knee-joint bandage.

The central area 30 has a greater elasticity than the base body 10 and is designed to be hexagonal in the exemplary embodiment shown here. The elasticity of the central area 30 may be designed to be greater in the mediolateral direction than in the proximal-distal direction, so that the joint bandage 1 is able to expand more easily in the area of the bend in the circumferential direction than in the longitudinal direction. The central area 30 is surrounded by transitional areas 20, which completely surround the central area 30 in the exemplary embodiment shown here. The transitional areas 20 are made of a highly elastic mesh-like textile, e.g., a knit having a greater elasticity than the central area 30. For the transitional areas 20, different elasticities may also be provided in different directions of stretching, but there is also the possibility of having the same elasticity in both the mediolateral direction and the proximal-distal direction.

Elasticity is understood to be the measure by which the textile can be lengthened without being destroyed. If a piece of textile can be doubled in extent by pulling in one direction, then it has an elasticity of 100%. The elasticity in the longitudinal extent of the base body of the bandage, i.e., in the proximal-distal direction, is between 80% and 130%, for example, while that of the transitional area 20 is between 120% and 180% and that of the central area 30 is between 100% and 140%. In the circumferential direction, i.e., in the mediolateral direction, elasticities between 180% and 220% may be provided, but the elasticity in the circumferential direction is preferably greater than that in the longitudinal direction. The elasticity in the area of the transitional area 20 and the central area 30 in the circumferential direction is achieved by omitting the weft fibers.

The hexagonal central area 30 has a flattened shape, where the connecting line between the two opposite corners that are the greatest distance apart essentially coincides with the axis of flexion, which is oriented approximately in the mediolateral direction. As an alternative to a hexagonal embodiment of the central area 30, it may also have a lenticular design.

The entire insert 2 has a biconvex outward curvature in the mediolateral direction, whereas it has a biconvex inward curvature in the direction of the central area 30 in the proximal-distal direction, thereby facilitating preflexion while preventing wrinkling in the flexed state. Because of the high elasticities in the area of the insert 2 and the preflexion of the bandage 1, it is possible that there is no bulging of material in the area of the bend in the joint with the bandage 1 applied, thereby preventing or at least reducing stress on the tendons or blood vessels.

The insert 2 may be incorporated into the base body 10, in particular being knit into it or incorporated in some other way in the production of the base body 10. The insert 2 simulates the behavior of the skin in flexion of a joint, such that the central area 30 having an elasticity greater than that of the base body 10 and smaller than that of the transitional areas 20 ensures induction of the compressed joint state at the start of flexion.

The transitional areas 20 prevent wrinkling and produce a tight contact of the central area 30 in the flexion of the joint, so a therapeutic pressure may also be applied to the tissue in the bend in the joint at the same time.

It is provided that the elasticity of the base body 10 increases incrementally in the transfer zones 4 distally and proximally of the insert 2, e.g., by only providing a weft thread in every second or third row of the knit. Likewise, transfer zones may also be provided medially and/or laterally of the insert 2 in order to provide a smooth transition from the relatively fixed base body 10 to the highly elastic transitional area 20 and the more highly elastic central area 30 in comparison with the base body 10.

1. A joint bandage having a tubular base body of an elastic textile material with an insert in the area of the bend in the joint, such that the elasticity of the insert is greater than that of the base body, characterized in that two areas (20, 30) of differing elasticity are formed in the insert (2), their individual elasticities being greater than that of the base body (10).

2. The joint bandage according to claim 1, characterized in that a central area (30) is formed in the insert (2) and is connected to transitional areas (20) at least medially and laterally, each of these transitional areas having a greater elasticity than the central area (30).

3. The joint bandage according to claim 2, characterized in that the central area (30) is designed to be essentially hexagonal or lenticular.

4. The joint bandage according to claim 2, characterized in that the elasticity of the central area (30) is greater in the mediolateral direction than in the proximal-distal direction.

5. The joint bandage according to claim 2, characterized in that the longest axis of the central area (30) lies in the mediolateral plane.

6. The joint bandage according to claim 2, characterized in that the transitional areas (20) have a mesh structure.

7. The joint bandage according to claim 2, characterized in that the elasticity of the transitional area (20) in the mediolateral direction is greater than that in the proximal-distal direction.

8. The joint bandage according to claim 2, characterized in that the central area (30) is incorporated into the transitional areas (20).

9. The joint bandage according to claim 1, characterized in that the edges of the insert (2) are designed with a curvature, such that the edges have an inward curvature in the proximal-distal extent and an outward curvature in the mediolateral extent.

10. The joint bandage according to claim 1, characterized in that the insert (2) is incorporated into the base body (10).

11. The joint bandage according to claim 1, characterized in that the joint bandage (1) is preflexed.

12. The joint bandage according to claim 1, characterized in that the joint bandage (1) is designed as a knit fabric.
